(19) United States
(12) Patent Application Publication
(10) Pub. No.: US 2018/0092960 A1
(43) Pub. Date: Apr. 5, 2018

(54) Method of Modulating the Concentration of Proteins in Cerebrospinal Fluid

(71) Applicant: Richard L. Bowen, Charleston, SC (US)

(72) Inventors: Richard L. BOWEN, Mount Pleasant, SC (US); Craig S. ATWOOD, Madison, WI (US)

(21) Appl. No.: 15/723,686

(22) Filed: Oct. 3, 2017

Related U.S. Application Data
(60) Provisional application No. 62/403,464, filed on Oct. 3, 2016.

(51) Int. Cl. A61K 38/09; A61K 39/395; C07K 16/26; C07K 16/28

(52) U.S. Cl. CPC A61K 38/09; A61K 39/3955; A61K 2039/505; C07K 16/2869; C07K 2317/76; C07K 16/26

(57) ABSTRACT

Method of modulating the concentrations of tau, P-tau, β-amyloid, NF-L, α-synuclein and/or neurogranin in cerebrospinal fluid (CSF) to prevent, stabilize and/or reverse suitable health conditions.

### CROSS REFERENCE TO RELATED APPLICATIONS

This application is a Non-provisional patent application and claims priority to U.S. Provisional Application Ser. No. 62/403,464, filed on Oct. 3, 2016, titled “METHOD OF MODULATING THE CONCENTRATION OF PROTEINS IN CEREBROSPINAL FLUID”, the disclosure of which is incorporated herein by reference in its entirety.

### FIELD OF THE INVENTION

The present invention concerns methods for altering the concentrations or ratios of specific proteins, primarily; tau, phosphorylated tau, β-amyloid and other cerebrospinal fluid proteins, in the cerebrospinal fluid of humans and other mammals.

### BACKGROUND OF THE INVENTION

Numerous unwanted health conditions are associated with abnormal concentrations or abnormal ratios of tau, phosphorylated tau, and β-amyloid, and other CSF proteins in the cerebrospinal fluid.

Accordingly, it is desirable to provide method of altering the concentrations or ratios of these proteins to those found in individuals without these unwanted health conditions.

### SUMMARY OF THE INVENTION

The foregoing needs are met, to a great extent, by the present invention, wherein a method of altering the concentrations of specific proteins, including tau, phosphorylated tau (P-tau), β-amyloid, NF-L, total α-synuclein, and/or neurogranin to more healthy levels thereby preventing, minimizing, reversing, or stabilizing unwanted health conditions including loss of brain function and other conditions associated with abnormal concentrations or ratios of these proteins that is capable of overcoming the disadvantages described herein at least to some extent is provided.

An embodiment of the present invention relates to a method of treating age-related loss in brain function by lowering CSF tau and/or P-tau by inhibiting gonadotropin signaling.

Another embodiment of the present invention pertains to a method of treating an imbalance of tau P-tau, NF-L, total α-synuclein and/or neurogranin in CSF by inhibiting gonadotropin signaling.

Yet another embodiment of the present invention relates to a method of treating age-related loss in brain function by administering a GnRH analog to a patient in need thereof.

Yet another embodiment of the present invention pertains to a method of treating age-related loss in brain function by administering leuprolide and its pharmaceutically acceptable salts to a patient in need thereof.

Yet another embodiment of the present invention relates to a method of treating age-related loss in brain function by raising the CSF concentration of β-amyloid to the upper most quartile and/or lowering the CSF concentration of tau P-tau, NF-L, total α-synuclein and/or neurogranin to the lowest quartile in a patient in need thereof.

Yet another embodiment of the present invention pertains to a method of treating age-related loss in brain function by raising the CSF concentration of β-amyloid relative to the CSF concentration of tau P-tau, NF-L, total α-synuclein and/or neurogranin in a patient in need thereof.

Yet another embodiment of the present invention relates to a method of preventing age-related loss in brain function by lowering CSF tau P-tau, NF-L, total α-synuclein and/or neurogranin by inhibiting gonadotropin signaling.

Yet another embodiment of the present invention pertains to a method of preventing age-related loss in brain function by administering a GnRH analog to a patient in need thereof.

Yet another embodiment of the present invention relates to a method of preventing age-related loss in brain function by administering leuprolide and its pharmaceutically acceptable salts to a patient in need thereof.

Yet another embodiment of the present invention pertains to a method of preventing age-related loss in brain function by raising the CSF concentration of β-amyloid to the upper most quartile and/or lowering the CSF concentration of tau P-tau, NF-L, total α-synuclein and/or neurogranin to the lowest quartile in a patient in need thereof.

Yet another embodiment of the present invention relates to a method of preventing age-related loss in brain function by raising the CSF concentration of β-amyloid relative to the CSF concentration of tau P-tau, NF-L, total α-synuclein and/or neurogranin in a patient in need thereof.

Yet another embodiment of the present invention pertains to a method of treating loss of brain function of a patient by lowering CSF tau P-tau, NF-L, total α-synuclein and/or neurogranin by inhibiting gonadotropin signaling.

Yet another embodiment of the present invention relates to a method of treating loss of brain function of a patient by administering a GnRH analog to a patient in need thereof.

Yet another embodiment of the present invention pertains to a method of treating loss of brain function of a patient by administering leuprolide and its pharmaceutically acceptable salts to a patient in need thereof.

Yet another embodiment of the present invention relates to a method of treating loss of brain function of a patient by raising the CSF concentration of β-amyloid to the upper most quartile and/or lowering the CSF concentration of tau P-tau, NF-L, total α-synuclein and/or neurogranin to the lowest quartile in a patient in need thereof.

Yet another embodiment of the present invention pertains to a method of treating loss of brain function of a patient by raising the CSF concentration of β-amyloid relative to the CSF concentration of tau P-tau, NF-L, total α-synuclein and/or neurogranin in a patient in need thereof.

Yet another embodiment of the present invention relates to a method of preventing loss of brain function of a patient by lowering CSF tau P-tau, NF-L, total α-synuclein and/or neurogranin by inhibiting gonadotropin signaling.

Yet another embodiment of the present invention pertains to a method of preventing loss of brain function of a patient by administering a GnRH analog to a patient in need thereof.

Yet another embodiment of the present invention relates to a method of preventing loss of brain function of a patient by administering leuprolide and its pharmaceutically acceptable salts to a patient in need thereof.

Yet another embodiment of the present invention pertains to a method of preventing loss of brain function of a patient by raising the CSF concentration of β-amyloid to the upper most quartile and/or lowering the CSF concentration of tau P-tau, NF-L, total α-synuclein and/or neurogranin to the lowest quartile in a patient in need thereof.

Yet another embodiment of the present invention relates to a method of preventing loss of brain function of a patient by raising the CSF concentration of β-amyloid relative to the CSF concentration of tau P-tau, NF-L, total α-synuclein and/or neurogranin in a patient in need thereof.

There has thus been outlined, rather broadly, certain embodiments of the invention in order that the detailed description thereof may be better understood herein, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### DETAILED DESCRIPTION

Embodiments of the invention provide a method of altering the concentrations or concentration ratios of specific proteins, primarily; tau, P-tau, β-amyloid, NF-L, total α-synuclein and/or neurogranin in the cerebrospinal fluid of humans and other mammals thereby preventing, reducing, stabilizing and or treating age-related loss in brain function (cognitive, motor and/or other) via the administration of a substance that decreases: gonadotropin releasing hormone GnRH its activity, and/or signaling; and/or gonadotropins, their activity, and/or signaling. It is believed that GnRH and/or gonadotropins stimulate an undesirable neuronal cell division in some instances and that within the cell division process tau protein is phosphorylated. Numerous drugs have been developed that inhibit the production of gonadotropins. As described herein, administering a substance that decreases GnRH and or gonadotropin concentrations and or production and or activity to a patient in need thereof will increase cerebrospinal fluid (CSF) concentrations of β-amyloid and/or decrease CSF concentrations of tau and/or P-tau. The concentration of tau and P-tau relative to β-amyloid in CSF may increase as a result of increasing levels of GnRH and or luteinizing hormone (LH) and or other gonadotropins. This increase in the concentration of tau and P-tau relative to β-amyloid in CSF may result in decreased cognitive function and/or loss of brain function. Even in the absence of dementia there is an age-related loss of brain (cognitive and/or motor) function. Rarely does anyone function as well cognitively at age 85 as they did at age 35. This “normal” age-related loss of brain function typically begins around age 50. Therefore, at age 40 individuals whose β-amyloid is below the upper most quartile would benefit to have it raised to as close to the upper most quartile as possible. Those whose tau or P-tau concentrations are above the lowest quartile would benefit to have them lowered to as close to the lowest quartile as possible.

Tau protein is a highly soluble microtubule-associated protein (MAP). These proteins are found mostly in neurons compared to non-neuronal cells. One of tau's main functions is to modulate the stability of axonal microtubules. Tau proteins interact with tubulin to stabilize microtubules and promote tubulin assembly into microtubules. It is active primarily in the distal portions of axons where it provides microtubule stabilization but also flexibility as needed. When serine or threonine tau residues are phosphorylated the protein dissociates from the tubules. Increased hyperphosphorylated tau proteins are found in the extracellular fluid of patients with neurodegenerative diseases as well as those with significant head trauma. Importantly, patients with head trauma that had high initial tau levels experienced worse clinical outcomes. Increased phosphorylated tau is also associated with advanced age.

More particularly, as used herein, the term, “age-related loss of cognitive function” refers to a measurable loss in cognitive function as the patient ages. In a particular example, if the patient performance in a cognitive test declines as the patient ages and the patient is otherwise healthy and does not show signs of dementia, it can be said that the patient suffers from age-related loss of cognitive function. The term, “treating age-related loss of cognitive function” as used herein, refers to slowing or preventing the progression of age-related loss of cognitive function or improving cognitive function.

In addition, the term, “person susceptible to age-related loss of cognitive function” generally refers to all aging patients and, more particularly, to individuals whose amyloid is below the upper most quartile or whose tau or P-tau concentrations are above the lowest quartile. In these patients, it may be possible to prevent age-related loss of cognitive function by raising the CSF concentration of amyloid to the upper most quartile and/or lowering the CSF concentration of tau and/or P-tau to the lowest quartile.

In addition to person susceptible to age-related loss of cognitive function, aspects of this invention may be useful in treating and/or preventing tauopathies and amyloidopathies in patients. Tauopathies are a class of neurodegenerative diseases associated with the pathological aggregation of tau protein in neurofibrillary or gliofibrillary tangles in the human brain. Tangles are formed by hyperphosphorylation of a microtubule-associated protein known as tau, causing it to aggregate in an insoluble form. Examples of tauopathies include: primary age-related tauopathy (PART)/Neurofibrillary tangle-predominant senile dementia; Lewy body dementia; dementia pugilistica; progressive supranuclear palsy; corticobasal degeneration; chronic traumatic encephalopathy “CTE”; frontotemporal dementia and parkinsonism linked to chromosome 17; lytico-Bodig disease (Parkinson-dementia complex of Guam); ganglioglioma and gangliocytoma; meningioangiomatosis; postencephalitic parkinsonism; subacute sclerosing panencephalitis; lead encephalopathy; tuberous sclerosis; Hallervorden-Spatz disease; lipofuscinosis; Down Syndrome, Autism, and the like. Examples of amyloidopathies include:

β-amyloid is a low molecular weight (39-43 amino acids) protein that is a proteolytic product derived from the larger amyloid precursor protein and is the major component of neocortical amyloid collections in Alzheimer's disease. As is the case with other amyloid proteins, β-amyloid originates as a normally soluble and constitutive protein found in biological fluids and tissue. β-amyloid also aggregates to form diffuse amorphous deposits in AD but also following head injury and in healthy aged individuals.

Parkinson's disease is associated with the presence of Lewy bodies. Clumps of specific substances within brain cells are microscopic markers of Parkinson's disease. These are called Lewy bodies, and researchers believe these Lewy bodies hold an important clue to the cause of Parkinson's disease.

Alpha-synuclein is found within Lewy bodies. Although many substances are found within Lewy bodies, scientists believe an important one is the natural and widespread protein called alpha-synuclein (α-synuclein). α-synuclein is found in all Lewy bodies in a clumped form that cells can't break down. This is currently an important focus among Parkinson's disease researchers. Parkinson's disease typically begins in middle or late life, and the risk increases with age. People usually develop the disease around age 60 or older. Men are more likely to develop Parkinson's disease than are women. Due to the fact that age and sex are the biggest risk factors we believe that stabilizing the HPG axis with Lupron will affect α-synuclein in the csf.

Neurofilaments are a major protein expressed within the axons in all neuronal cells. Their main function is to maintain the axonal calibre and they thereby possess a crucial role for morphological integrity and conduction velocity of nerve impulses. There are three neurofilament components in nerve fibres and they are classified according to their molecular weights as determined by SDS-PAGE i.e. neurofilament light (NF-L), neurofilament medium (NF-M) and neurofilament heavy (NF-H). The neurofilament light act as the backbone to which NF-M and NF-H copolymerise to form these intermediate filaments. These neurofilaments are released into the cerebrospinal fluid during trauma and in several neurodegenerative diseases. The presence of elevated levels of neurofilament light polypeptide (NF-L) in the CSF is an indicator of axonal damage in the CNS.

Neurogranin is a calmodulin-binding protein of 78 amino acids expressed exclusively in the brain, particularly in dendritic spines, and participating in the protein kinase C signaling pathway. Neurogranin is the main postsynaptic protein regulating the availability of calmodulin, binding to it in the absence of calcium. Phosphorylation by protein kinase C lowers its binding ability.

Neurogranin is a post-synaptic protein and a putative marker of synaptic loss in AD, an event which may be closely linked to development of cognitive decline. CSF neurogranin is increased in the early clinical stages of dementia and is increased in AD compared to other dementias. CSF neurogranin is associated with brain atrophy and reduced brain glucose uptake.

Some or all of the tauopathies and amyloidopathies may be evident in a patient as a loss of motor function. For the purposes of this disclosure, the term, ‘motor function’ refers to muscular coordination and/or muscular strength of the patient.

In addition to a patient susceptible to age-related loss of cognitive function and tauopathies, aspects of this invention may be useful in treating and/or preventing other undesirable conditions associated with loss of brain function where CSF concentrations of β-amyloid are lower than normal and/or tau and/or P-tau are higher than normal. For example, loss of cognitive function may result from general anesthesia, by-pass surgery, CTE, chemotherapy, etc.

Detection of increased amyloid tissue deposition by PET and decreased brain volume by MM are associated with lower CSF amyloid and higher tau and phospho-tau concentrations. Therefore, we believe that raising CSF amyloid and lowering tau phospho-tau concentrations will result in a decrease or stabilization of amyloid deposition and an increase or stabilization of brain volume.

In accordance with an embodiment of the invention, age-related loss of cognitive function, tauopathies, other undesirable conditions associated with loss of brain function where CSF concentrations of β-amyloid are lower than normal and/or tau and/or P-tau are higher than normal in a patient susceptible to or suffering therefrom can be treated by administration to the patient any composition that increases in the concentration of β-amyloid relative to tau and P-tau in CSF, in an amount and for a duration effective to bring about such a change.

In accordance with another embodiment of the invention, age-related loss of cognitive function, tauopathies, and other undesirable conditions associated with loss of brain function where CSF concentrations of β-amyloid are lower than normal and/or tau and/or P-tau are higher than normal in a patient susceptible to or suffering therefrom can be treated by administration to the patient of any composition that increases the concentration of β-amyloid in CSF, in an amount and for a duration effective to bring about such an increase.

In accordance with yet another embodiment of the invention, age-related loss of cognitive function, tauopathies, and other undesirable conditions associated with loss of brain function where CSF concentrations of β-amyloid are lower than normal and/or tau and/or P-tau are higher than normal in a patient susceptible to or suffering therefrom can be treated by administration to the patient of any composition that decreases the concentration of tau and/or P-tau in CSF, in an amount and for a duration effective to bring about such a decrease.

Further, in accordance with the invention, age-related loss of cognitive function, tauopathies, and other undesirable conditions associated with loss of brain function where CSF concentrations of β-amyloid are lower than normal and/or tau and/or P-tau are higher than normal can be prevented, or onset of clinical or behavioral manifestations delayed, in a patient susceptible to age-related loss of cognitive function by administration to the patient of any composition that: increases in the concentration of β-amyloid relative to tau and P-tau in CSF; increases the concentration of β-amyloid in CSF; and/or decreases the concentration of tau and/or P-tau in CSF, in an amount and for a duration effective to bring about such a change to a level below which development of the age-related loss of cognitive function will not occur.

Reference herein to “level of a hormone” in a patient means concentration of the biologically active hormone in the patient's CSF, extracellular fluid, and/or serum. Typically, the level of a hormone will be reduced by reducing the concentration of the hormone itself. However, reducing the activity of the hormone—as, for example, by binding it with an antibody that blocks the hormone's activity—even if the concentration of the hormone remains the same, is considered reducing the level of the hormone for purposes of the present application. The serum concentrations of biologically active FSH and LH in a human can be determined by any of a number of methods well known to the skilled.

As understood in the art, vaccines that stimulate production of antibodies can be employed to bind to FSH, LH, or GnRH and block or at least substantially reduce their biological activities. Thus, vaccine-stimulated antibodies to FSH, LH or both can be employed in accordance with the invention to directly reduce the level of these hormones and thereby treat or prevent age-related loss of cognitive function, tauopathies, and other undesirable conditions associated with loss of brain function where CSF concentrations of β-amyloid are lower than normal and/or tau and/or P-tau are higher than normal. Such antibodies to GnRH, by blocking its activity, will result in reduced levels of FSH and LH. These antibodies can be employed in accordance with the invention to reduce levels of GnRH, FSH and LH, or FSH or LH alone, and thereby to prevent or treat age-related loss of cognitive function, tauopathies, and other undesirable conditions associated with loss of brain function where CSF concentrations of β-amyloid are lower than normal and/or tau and/or P-tau are higher than normal.

Antibodies for use in accordance with the invention may be made by conventional methods for preparation of vaccine antibodies for therapeutic use in humans. The vaccine-stimulated antibodies may be polyclonal and from any antibody-producing species, such as mice, rats, horses, dogs or humans. The antibodies may also be, and preferably are, monoclonal from cultures of antibody-producing cells from an antibody-producing species such as mice, rats, horses, dogs, and humans. The term “antibody” as used herein, unless otherwise limited, also encompasses antigen-binding fragments, such as Fab fragments, of intact antibodies. If an antibody is monoclonal but from cultured cells of a species other than human, the antibody may be “humanized” by conventional methods to make it more tolerable immunologically to a person treated therewith. Antibodies for use in accordance with the invention can also be made by conventional techniques using cultured cells, preferably human cells, that have been genetically engineered to make a desired intact antibody or antigen-binding antibody fragment.

Antibodies will be administered in accordance with the invention by any method known in the art for administering same but preferably by intravenous injection of a sterile aqueous solution of the antibody, together with standard buffers, preservatives, excipients and the like.

In general, it has been unexpectantly found that compounds that antagonize the activity of GnRH or stimulate production of inhibin in the production of FSH and LH are capable of increasing in the concentration of β-amyloid relative to tau and P-tau in CSF. Thus, compounds that block the receptors for GnRH or stimulate receptors of inhibin on the pituitary, or that otherwise act on the pituitary-ovarian or pituitary-testicular axis to inhibit production or activity of GnRH or stimulate production of or activate inhibin, or to directly inhibit production of FSH or LH or both, will result in reduced levels of FSH and LH and can be employed in accordance with the invention to increase in the concentration of β-amyloid relative to tau and P-tau in CSF to treat or prevent age-related loss of cognitive function, tauopathies, and other undesirable conditions associated with loss of brain function where CSF concentrations of β-amyloid are lower than normal and/or tau and/or P-tau are higher than normal. One such compound is danazol (see The Merck Index, Merck & Co., Inc., Whitehouse Station, N.J., U.S.A 12th Ed. 1996 (hereinafter “Merck Index”), entry no. 2875, and U.S. Pat. No. 3,135,743). Such compounds, also, will be administered by any standard route as understood in the art.

As indicated above, GnRH analogs (or physiologically acceptable salts thereof) and inhibin analogs (or physiologically acceptable salts thereof) can also be used in accordance with the invention to treat or prevent age-related loss of cognitive function, tauopathies, and other undesirable conditions associated with loss of brain function where CSF concentrations of β-amyloid are lower than normal and/or tau and/or P-tau are higher than normal by increasing in the concentration of β-amyloid relative to tau and P-tau in CSF. Of these GnRH analogs, most preferred is leuprolide or goserelin, and especially leuprolide acetate and goserelin acetate.

In a specific example, leuprolide acetate is given to the patient to increase the concentration of β-amyloid in CSF, and/or decrease the concentration of tau and/or P-tau in CSF, and/or increase in the concentration of β-amyloid relative to tau and P-tau in CSF.

GnRH analogs or salts thereof that may be employed in accordance with the invention include, among others, GnRH itself and its monoacetate and diacetate salt hydrates (Merck Index entry no. 5500) and the many analogs thereof that are known in the art. These include, for example, leuprolide and its monoacetate salt (Merck Index entry no. 5484, U.S. Pat. No. 4,005,063); the analogs of leuprolide with the D-leucyl residue replaced with D-α-aminobutyryl, D-isoleucyl, D-valyl or D-alanyl and the monoacetate salts thereof (U.S. Pat. No. 4,005,063); buserelin and its monoacetate salt (Merck Index entry no. 1527, U.S. Pat. No. 4,024,248); nafarelin and its monoacetate and acetate hydrate salts (Merck Index entry no. 6437, U.S. Pat. No. 4,234,571); deslorelin (Merck Index entry no. 2968); histrelin and its acetate salt (Merck Index entry no. 4760, U.S. Pat. No. 4,244,946); and goserelin and its acetate salt (Merck Index entry no. 4547, U.S. Pat. No. 4,100,274). For other GnRH analogs and salts thereof that can be used in accordance with the invention, see also U.S. Pat. No. 4,075,192, U.S. Pat. No. 4,762,717, and the U.S. patents cited at column 3, lines 49-54, of U.S. Pat. No. 4,762,717.

All of the U.S. patents cited herein, including those not cited specifically but cited at column 3, lines 49-54, of U.S. Pat. No. 4,762,717, and all of the Merck Index entries cited herein are incorporated herein by reference.

GnRH analogs work by over stimulating the GnRH receptor causing an initial increase in signaling followed by a complete down regulation of the receptor and its signaling. Whereas with inhibin analogs, the stimulation or increase in inhibin signaling decreases GnRH. Administration of GnRH analogs, and inhibin analogs, in accordance with the invention will be by any method known in the art for administering same. Thus, administration may be by injection subcutaneously, intramuscularly or intravenously of a sterile aqueous solution which includes the analog together with buffers (e.g., sodium acetate, phosphate), preservatives (e.g., benzyl alcohol), salts (e.g., sodium chloride) and possibly various excipients or carriers. In this connection, see, for example, Physician's Desk Reference, 51st Ed., Medical Economics Co., Montvale, N.J., U.S.A. (1997), pp. 2736-2746 (leuprolide acetate) and pp. 2976-2980 (goserelin acetate), which are also incorporated herein by reference.

The dose and dosage regimen for a particular composition used to carry out the invention with a particular patient will vary depending on the active (i.e., LH-lowering or FSH-lowering) ingredient and its concentration and other components in the composition, the route of administration, the gender, age, weight, and general medical condition of the individual, and whether the patient is already suffering from age-related loss of cognitive function, tauopathies, and other undesirable conditions associated with loss of brain function where CSF concentrations of β-amyloid are lower than normal and/or tau and/or P-tau are higher than normal. The skilled medical practitioner will be able to appropriately prescribe dosage regimens to carry out the invention. It is preferred in carrying out the invention that the CSF concentration of β-amyloid is raised to the upper most quartile and/or the CSF concentration of tau and/or P-tau is lowered to the lowest quartile. However, beneficial effects of preventing or reducing susceptibility to age-related loss of cognitive function, tauopathies, and other undesirable conditions associated with loss of brain function where CSF concentrations of β-amyloid are lower than normal and/or tau and/or P-tau are higher than normal, or treating age-related loss of cognitive function, tauopathies, and other undesirable conditions associated with loss of brain function where CSF concentrations of β-amyloid are lower than normal and/or tau and/or P-tau are higher than normal are achieved even if the concentration of β-amyloid is raised relative to the concentration of tau and/or P-tau. Thus, the medical practitioner will select the composition, dose and dosage regimen for a particular patient to achieve and maintain relatively concentrations of β-amyloid, low concentrations of tau and/or P-tau or both in the individual.

In carrying out the invention, compounds that block the receptors for GnRH or stimulate receptors of inhibin on the pituitary or other suitable inhibin receptors, or that otherwise act on the pituitary-ovarian or pituitary-testicular axis to inhibit production or activity of GnRH or stimulate production of or activate inhibin, or to directly raise the concentration of amyloid relative to tau and/or P-tau, are administered at between about 0.001 g and 10 g per day.

In a most preferred embodiment of carrying out the invention, a composition comprising a GnRH analog will be administered intramuscularly or subcutaneously as a depot composition from which release of the analog into the patient's system will be sustained over a long period, from about a week to about six months or more. This will raise or maintain the concentration of amyloid relative to tau and/or P-tau in the patient as described above without the pain, cost and inconvenience of much more frequent (e.g., daily) administration. Such depot compositions of GnRH analogs are known and their preparation is well within the skill of the ordinarily person skilled in the art. See, e.g., Physician's Desk Reference, 51st Ed. pp. 2736-2746 and 2976-2980, cited above.

To allow the skilled medical practitioner to easily establish doses and dosage regimens of GnRH analogs for treating or preventing age-related loss of cognitive function, tauopathies, and other undesirable conditions associated with loss of brain function where CSF concentrations of β-amyloid are lower than normal and/or tau and/or P-tau are higher than normal in a patient in accordance with the invention, doses and dosage regimens for goserelin acetate and leuprolide acetate are provided here. Doses of goserelin acetate effective to treat or prevent age-related loss of cognitive function, tauopathies, and other undesirable conditions associated with loss of brain function where CSF concentrations of β-amyloid are lower than normal and/or tau and/or P-tau are higher than normal range from about 3 mg to about 90 mg, preferably about 10-30 mg, with a once monthly subcutaneous injection of a sterile depot formulation of from about 60 mg to about 90 mg per month, preferably about 30 mg to 60 mg, with a subcutaneous injection once every three months of a sterile depot formulation.

Doses of leuprolide acetate effective to treat or prevent age-related loss of cognitive function, tauopathies, and other undesirable conditions associated with loss of brain function where CSF concentrations of β-amyloid are lower than normal and/or tau and/or P-tau are higher than normal range between about 0.2 and 20 mg/day, preferably about 1 mg/day, when the dosage regimen is by once daily, subcutaneous injection of sterile solution comprising the compound; between about 1 mg and about 10 mg, preferably about 5 mg, with a once monthly intramuscular injection of a sterile depot formulation comprising the compound; and between about 10 mg and about 50 mg, preferably about 25 mg, with an intramuscular injection once every three months of a depot formulation comprising the compound.

Information from data already available or easily obtained by routine experimentation on GnRH analogs in suppressing GnRH, LH and FSH activity, those of ordinary skill can easily determine the dose and dosage regimens for any GnRH analog.

It is preferred that treatment in accordance with the invention is administered with minimal interruption for the duration of the patient's life. The reason for this uninterrupted administration is that, once such administration is discontinued, the persons' natural production of GnRH, FSH and LH will resume within at most a few months or, more typically, within a few weeks.

In the following examples, methods of lowering CSF tau and/or P-tau by inhibiting gonadotropin signaling are provided. However, it is within the scope and spirit of the invention that the concentration of β-amyloid is raised relative to tau and/or P-tau in any suitable manner.

### METHODS

### Example 1

### Modulating CSF Protein Concentrations in Humans Using GnRH Agonists

Female patients (n=20) that are post-menopausal and neurologically normal are subjected to a lumbar puncture to collect CSF. The CSF is analyzed to determine a baseline concentration for CSF proteins. The patients are then injected with a GnRH superagonist (leuprolide acetate; Lupron Depot). Another Lupron Depot injection is given to the patients after one month. At 3 months another lumbar puncture is performed to collect CSF to compare concentrations of β-amyloid, tau, P-tau, NF-L, total α-synuclein and/or neurogranin to baseline levels. Expected results are that CSF β-amyloid will remain the same or increase while tau and P-tau will decrease.

### Example 2

### Modulating CSF Protein Concentrations in Humans Using GnRH Antagonists

Female patients are treated with a GnRH antagonist (i.e. Cetrorelix, Ganirelix, Abarelix, Degarelix) and CSF proteins assessed as described in Example 1.

### Example 3

### Modulating CSF Protein Concentrations in Humans Using Antibodies Against Gonadotropins

Female patients are treated with an LH receptor- and/or FSH receptor-blocking antibody and CSF proteins assessed as described in Example 1.

### Example 4

### Modulating CSF Protein Concentrations in Animals Using Modulators of GnRH and Gonadotropins

A statistically relevant number of canine that are neurologically normal, ages 11 years and above are used to determine if tau and P-tau can be lowered in CSF. A lumbar puncture or some other procedure is performed to collect CSF. The CSF is analyzed to determine a baseline. The subjects are then injected with Lupron Depot. Another Lupron Depot injection is given to the subjects after one month. At 3 months another lumbar puncture is performed to collect CSF and compare concentrations of β-amyloid, tau, and P-tau to baseline levels. Expected results are that CSF β-amyloid will remain the same or increase while tau, P-tau, NF-L, total α-synuclein and neurogranin will decrease.

As is demonstrated in examples 1-4 described herein, the relative concentration of β-amyloid to tau/P-tau or the other CSF proteins may be increased by modulating GnRH and gonadotropin signaling.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A method of treating age-related loss in brain function comprising modulating concentrations or concentration ratios of specific proteins, including: tau; P-tau; β-amyloid; NF-L; total α-synuclein; and/or neurogranin in the cerebrospinal fluid of a patient in need thereof.

2. The method according to claim 1, further comprising:
lowering CSF tau and/or P-tau by inhibiting gonadotropin signaling.

3. The method according to claim 1, further comprising:
administering a GnRH analog to a patient in need thereof;

4. The method according to claim 1, further comprising:
administering leuprolide and its pharmaceutically acceptable salts to a patient in need thereof.

5. The method according to claim 1, further comprising:
raising the CSF concentration of β-amyloid to the upper most quartile and/or lowering the CSF concentration of tau P-tau, NF-L, total α-synuclein and/or neurogranin to the lowest quartile in a patient in need thereof.

6. The method according to claim 1, further comprising:
raising the CSF concentration of β-amyloid relative to the CSF concentration of tau P-tau, NF-L, total α-synuclein and/or neurogranin in a patient in need thereof.

7. A method of preventing age-related loss in brain function by lowering CSF tau P-tau, NF-L, total α-synuclein and/or neurogranin by inhibiting gonadotropin signaling.

8. The method according to claim 7, further comprising: administering a GnRH analog to a patient in need thereof.

9. The method according to claim 7, further comprising: administering leuprolide and its pharmaceutically acceptable salts to a patient in need thereof.

10. The method according to claim 7, further comprising: raising the CSF concentration of β-amyloid to the upper most quartile and/or lowering the CSF concentration of tau P-tau, NF-L, total α-synuclein and/or neurogranin to the lowest quartile in a patient in need thereof.

11. The method according to claim 7, further comprising: raising the CSF concentration of β-amyloid relative to the CSF concentration of tau P-tau, NF-L, total α-synuclein and/or neurogranin in a patient in need thereof.

12. A method of treating loss of brain function of a patient by lowering CSF tau P-tau, NF-L, total α-synuclein and/or neurogranin by inhibiting gonadotropin signaling.

13. The method according to claim 12, further comprising: administering a GnRH analog to a patient in need thereof.

14. The method according to claim 12, further comprising: administering leuprolide and its pharmaceutically acceptable salts to a patient in need thereof.

15. The method according to claim 12, further comprising: raising the CSF concentration of β-amyloid to the upper most quartile and/or lowering the CSF concentration of tau P-tau, NF-L, total α-synuclein and/or neurogranin to the lowest quartile in a patient in need thereof.

16. The method according to claim 12, further comprising: raising the CSF concentration of β-amyloid relative to the CSF concentration of tau P-tau, NF-L, total α-synuclein and/or neurogranin in a patient in need thereof.
